(19) United States
(12) Patent Application Publication
(10) Pub. No.: US 2024/0156405 A1
(43) Pub. Date: May 16, 2024

(54) SIGNAL CALIBRATION METHOD AND RELATED SMART WEARABLE DEVICE

(71) Applicant: PixArt Imaging Inc., Hsin-Chu City (TW)

(72) Inventors: Yung-Chang Lin, Hsin-Chu City (TW); Jian-Cheng Liao, Hsin-Chu City (TW); Chun-Chih Chen, Hsin-Chu City (TW); Sen-Huang Huang, Hsin-Chu City (TW); Yen-Min Chang, Hsin-Chu City (TW)

(73) Assignee: PixArt Imaging Inc., Hsin-Chu City (TW)

(21) Appl. No.: 18/411,033

(22) Filed: Jan. 12, 2024

Related U.S. Application Data
(63) Continuation-in-part of application No. 17/835,930, filed on Jun. 8, 2022.
Application No. 17/835,930 is a continuation-in-part of application No. 18/118,152, filed on Mar. 7, 2023.
(60) Provisional application No. 63/357,009, filed on Jun. 30, 2022.

(30) Foreign Application Priority Data
Jun. 11, 2021 (CN) 202110656442.1
May 5, 2022 (CN) 202210481440.8
Dec. 27, 2022 (CN) 202211680498.1

(51) Int. Cl. A61B 5/00; A61B 5/0205; A61B 5/145

(52) U.S. Cl. CPC A61B 5/6843; A61B 5/0205; A61B 5/14542; A61B 5/6813; A61B 5/746; A61B 2560/0223

(57) ABSTRACT

A smart wearable device has a signal calibration function executed by a signal calibration method and applied to a finger, a limb and/or a neck of a user. The smart wearable device includes at least one physiological signal detector, at least one pressure detector and an operation processor. The at least one physiological signal detector is adapted to abut against a detection area of the user for detecting a physiological signal. The at least one pressure detector is disposed around the at least one physiological signal detector and adapted to detect a pressure value of the detection area. The operation processor is electrically connected with the at least one physiological signal detector and the at least one pressure detector. The operation processor is adapted to optimize the physiological signal when the pressure value exceeds a predefined pressure threshold.

### CROSS REFERENCE TO RELATED APPLICATIONS

This application is a continuation-in-part of U.S. application Ser. No. 17/835,930, filed on Jun. 8, 2022. Further, this application is a continuation-in-part of U.S. application Ser. No. 18/118,152, filed on Mar. 7, 2023, which claims the benefit of U.S. Provisional Application No. 63/357,009, filed on Jun. 30, 2022. The contents of these applications are incorporated herein by reference.

### BACKGROUND OF THE INVENTION

### 1. Field of the Invention

The present invention relates to a signal calibration method and a related smart wearable device, and more particularly, to a signal calibration method of calibrating a physiological signal affected by pressured and a related smart wearable device.

### 2. Description of the Prior Art

A conventional smart ring uses the optical detector to detect a blood oxygen level or a heart rate value of the user. In a long time of wearing the smart ring, the user may put heavy pressure on the smart ring due to changes in posture, such as sleeping or resting. When the user puts the heavy pressure on the smart ring by the palm, the skin of the finger is deformed, which results in reduction of a detection accuracy of the optical detector. If the smart ring sends out the incorrect physiological detection result frequently, the users' confidence in the product quality is affected accordingly. Therefore, design of a physiological detection device of optimizing the detection result in response to external pressure is an important issue in a related medical equipment industry.

### SUMMARY OF THE INVENTION

The present invention provides a signal calibration method and a related smart wearable device, and more particularly, to a signal calibration method of calibrating a physiological signal affected by pressured and a related smart wearable device for solving above drawbacks.

According to the claimed invention, a smart wearable device has a signal calibration function and applied to a finger, a limb and/or a neck of a user. The smart wearable device includes at least one physiological signal detector, at least one pressure detector and an operation processor. The at least one physiological signal detector is adapted to abut against a detection area of the user for detecting a physiological signal. The at least one pressure detector is disposed around the at least one physiological signal detector and adapted to detect a pressure value of the detection area. The operation processor is electrically connected with the at least one physiological signal detector and the at least one pressure detector. The operation processor is adapted to optimize the physiological signal when the pressure value exceeds a predefined pressure threshold.

According to the claimed invention, the at least one pressure detector is disposed adjacent to an outer edge of the at least one physiological signal detector, so that the physiological signal and the pressure value of the detection area are detected by the at least one physiological signal detector and the at least one pressure detector in a respective and simultaneous manner.

According to the claimed invention, the operation processor is adapted to further mark the physiological signal detected in a process of the pressure value exceeding the predefined pressure threshold, and output a warning reminder relevant to the marked physiological signal. Further, the operation processor is adapted to further replace the physiological signal detected in a process of the pressure value exceeding the predefined pressure threshold by the physiological signal detected before the process of the pressure value exceeding the predefined pressure threshold.

According to the claimed invention, the operation processor is adapted to further optimize the physiological signal when the pressure value exceeds the predefined pressure threshold and amplitude change of the physiological signal exceeds a predefined physiological threshold. Or, the operation processor is adapted to further optimize the physiological signal when the pressure value exceeds the predefined pressure threshold and duration of amplitude change of the physiological signal exceeding a predefined physiological threshold conforms to a predefined period of time. Or, the operation processor is adapted to further output a warning reminder relevant to the predefined pressure threshold when duration of the pressure value exceeding the predefined pressure threshold conforms to a predefined period of time.

According to the claimed invention, the smart wearable device further includes a plurality of physiological signal detectors and a plurality of pressure detectors, each of the plurality of pressure detectors is disposed adjacent to the outer edge of a corresponding physiological signal detector as a pair, for simultaneously detecting the physiological signal and the pressure value of a corresponding detection area.

According to the claimed invention, the smart wearable device further includes an acceleration detector electrically connected with the operation processor and adapted to detect an acceleration value of the detection area, the operation processor is adapted to further optimize the physiological signal when the pressure value exceeds the predefined pressure threshold and the acceleration value exceeds a predefined acceleration threshold.

According to the claimed invention, a signal calibration method of calibrating a physiological signal detected by a smart wearable device wore on a detection area of a user is disclosed. The signal calibration method includes analyzing a pressure value of the detection area detected by at least one pressure detector of the smart wearable device, and optimizing the physiological signal of the detection area detected by at least one physiological signal detector of the smart wearable device when the pressure value exceeds a predefined pressure threshold, for acquiring preferred accuracy of the physiological signal.

The smart wearable device of the present invention can utilize the detection results of the pressure detector and the acceleration detector to calibrate and/or optimize the physiological signal detected by the physiological signal detector; if the pressure value of the pressure detector and the acceleration value of the acceleration detector are at the high level, the smart wearable device may be set in an abnormal operation state, and the physiological signal generated within the related period can be marked as the abnormal result, or can be directly replaced by the computation value of the normal physiological signal. The pressure detector can be a capacitive pressure detector, a resistive pressure detector or a piezoelectric pressure detector; any pressure detector that can be disposed inside the case of the smart wearable device and provide the detection accuracy meeting the pressure standard can belong to a design scope of the present invention. The present invention can utilize both the pressure detector and the acceleration detector to optimize the detection result of the physiological signal detector, so as to effectively avoid the abnormal detection result caused by the user's posture, thus improving product credibility and reliability.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are sectional view of a smart wearable device according to different embodiments of the present invention.

FIG. 3 is a variation diagram of the physiological signal detected by the physiological signal detector 18 according to the embodiment of the present invention.

FIG. 4 is a flow chart of a signal calibration method according to the embodiment of the present invention.

### DETAILED DESCRIPTION

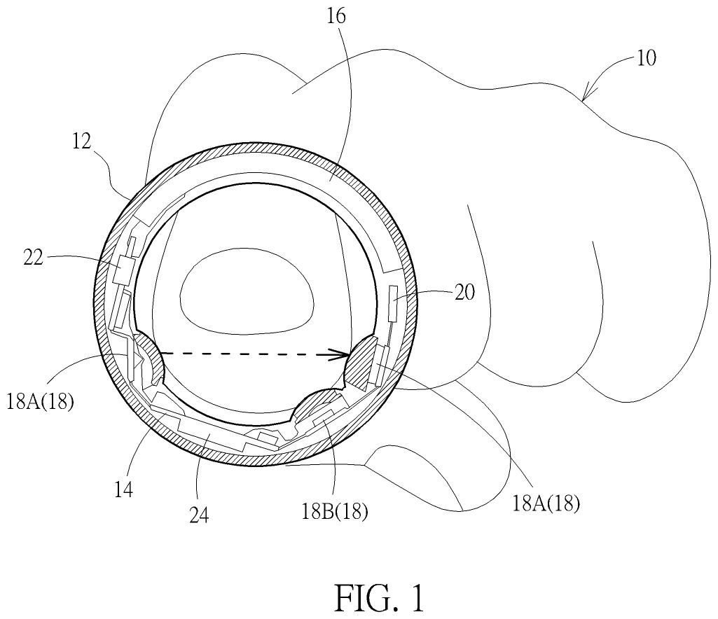

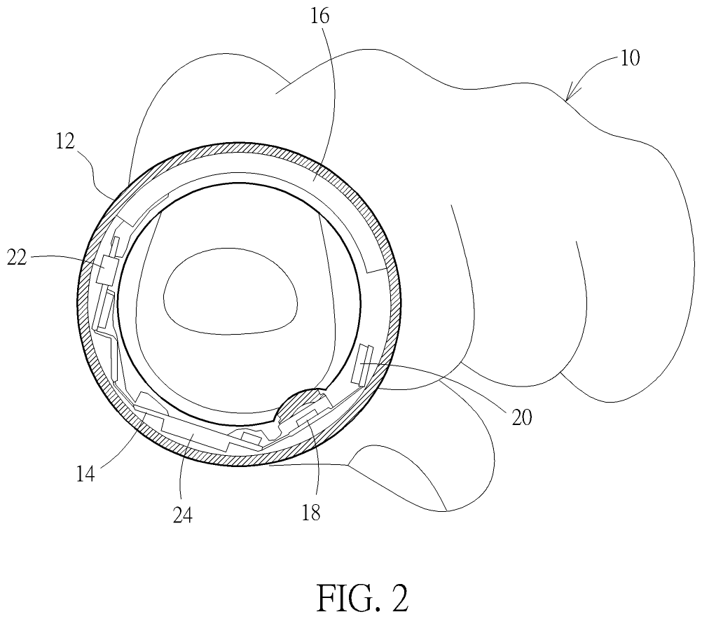

Please refer to FIG. 1 and FIG. 2. FIG. 1 and FIG. 2 are sectional view of a smart wearable device 10 according to different embodiments of the present invention. The smart wearable device 10 can be worn on a body of the user, such as a finger, a wrist, an arm, a forehead, a neck or a leg of the user, for detecting a physiological signal of the user. In the embodiments shown in FIG. 1 and FIG. 2, the smart wearable device 10 can be designed as a ring, and an application of the smart wearable device 10 is not limited to the foresaid type. In addition, the smart wearable device 10 can include several detectors used to detect an operation status of the user for deciding whether to actuate a signal calibration function of adjusting the detected physiological signal, so as to effectively increase detection accuracy of the smart wearable device 10.

The smart wearable device 10 can optionally include a case 12, a circuit board 14, a battery 16, a physiological signal detector 18, a pressure detector 20, an acceleration detector 22 and an operation processor 24. The case 12 can accommodate other electronic elements; as an example of the embodiment shown in FIG. 1 and FIG. 2, the smart wearable device 10 is designed as the ring, and the case 12 can be an annular shell with a radial side similar to the finger of the user. The circuit board 14 can be flexible circuit boards distributed in different locations within the case 12, or rigid circuit boards electrically connected with each other via cables; variation of the circuit board 14 can depend on a design demand. The battery 16 can supply power to other electronic elements.

A number of the physiological signal detector 18 can depend on the design demand. In the embodiment shown in FIG. 1, the smart wearable device 10 can include several physiological signal detectors 18, such as a transmissive-type physiological signal detector 18A and a reflective-type physiological signal detector 18B. The smart wearable device 10 may include single one physiological signal detector 18, such as the reflective-type physiological signal detector 18 shown in FIG. 2. Variation of a number and a type of the physiological signal detector 18 is not limited to the foresaid embodiments. In the present invention, the physiological signal detector 18 can contact against a detection area on the user body to detect the physiological signal, such as a blood oxygen level or a heart rate value. The detection area can be interpreted as a skin area of the user where on the smart wearable device 10 is worn.

The smart wearable device 10 may be worn on the user body for a long period. If the physiological signal detector 18 is pressed heavily due to posture change of the user, the detection accuracy of the physiological signal may be affected. Therefore, the present invention can dispose the pressure detector 20 around the physiological signal detector 18 to detect a pressure value of the detection area. A number of the pressure detector 20 can correspond to a number of the physiological signal detector 18. Each pressure detector 20 can be disposed adjacent to an outer edge of the corresponding physiological signal detector 18 as a pair, for detecting the physiological signal and the pressure value of the detection area in a respective and simultaneous manner.

Moreover, the present invention can optionally utilize the acceleration detector 22 to detect an acceleration value of the detection area. The operation processor 24 can be disposed on the circuit board 14 and electrically connected with the physiological signal detector 18, the pressure detector 20 and the acceleration detector 22. The operation processor 24 can immediately analyze the pressure value from the pressure detector 20 and the acceleration value from the acceleration detector 22, and can optimize the physiological signal when the pressure value and/or the acceleration value are abnormal, so as to improve the detection accuracy of the blood oxygen level or the heart rate value generated by the smart wearable device 10.

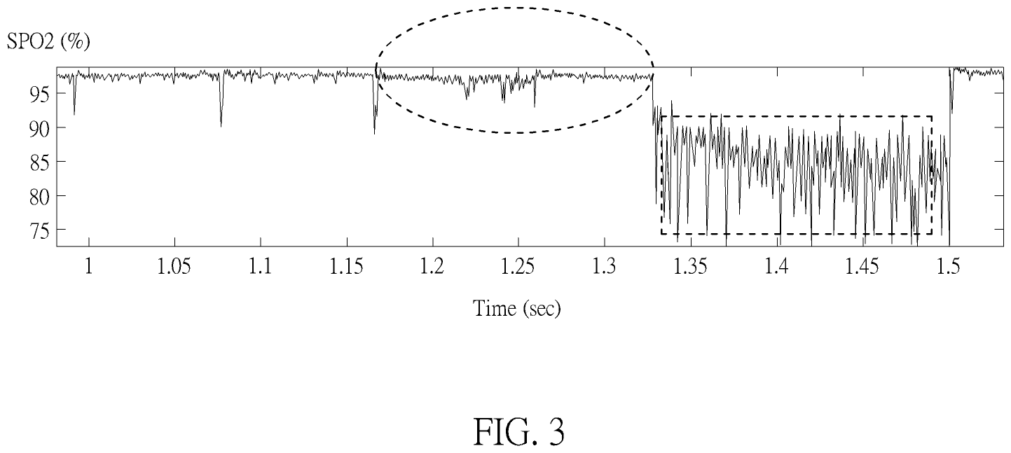

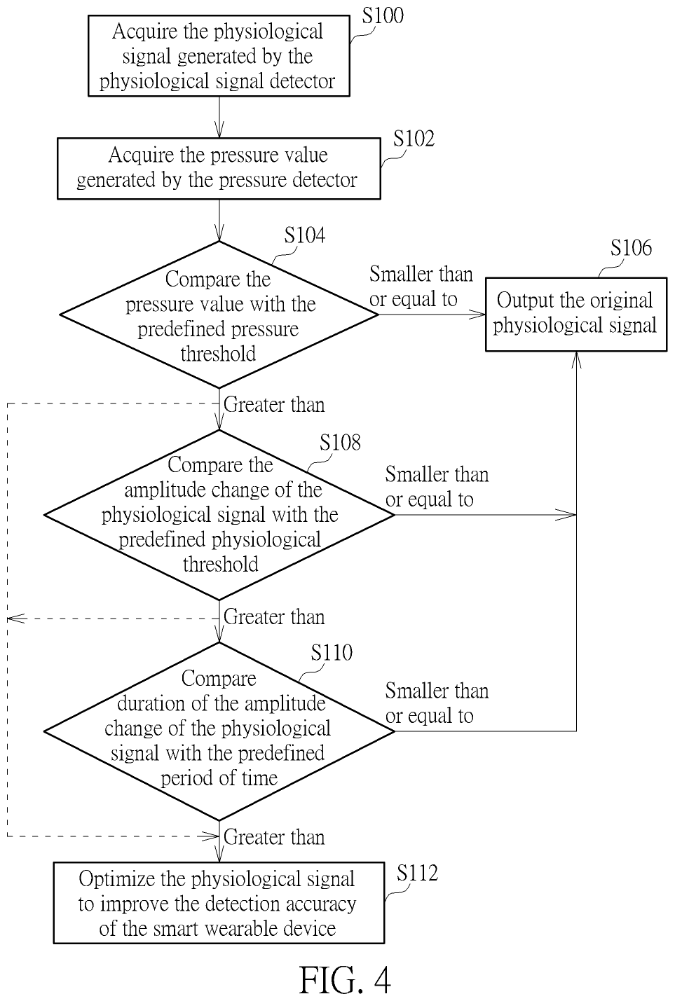

Please refer to FIG. 3 and FIG. 4. FIG. 3 is a variation diagram of the physiological signal detected by the physiological signal detector 18 according to the embodiment of the present invention. FIG. 4 is a flow chart of a signal calibration method according to the embodiment of the present invention. The signal calibration method illustrated in FIG. 4 can be suitable for the smart wearable device 10 shown in FIG. 1 and FIG. 2. As shown in FIG. 3, the physiological signals are at a high level in the early period of the detection process, and conform to the normal standard; in some later period, such as a dashed rectangular box, the physiological signals are changed drastically to fall into a low level, which means amplitude change of the physiological signal may exceed a predefined physiological threshold, so that the signal calibration method of the present invention can optimize the abnormal physiological signal in the foresaid later period.

Therefore, step S100 can be executed to acquire and store the original physiological signal generated by the physiological signal detector 18 which detects the detection area of the user body. Then, step S102 and step S104 can be executed to acquire the pressure value generated by the pressure detector 20 which detects the detection area, and compare the pressure value with the predefined pressure threshold. A value of the predefined pressure threshold can depend on detection sensitivity of the pressure detector 20 and a body feature of the user, and a detailed description is omitted herein for simplicity.

If the pressure value is smaller than or equal to the predefined pressure threshold, the smart wearable device 10 may be not under excessive stress, so step S106 can be executed to output the original physiological signal generated by the physiological signal detector 18. If the pressure value is greater than the predefined pressure threshold, the smart wearable device 10 may be under the excessive stress, and step S108 can be executed to compare the amplitude change of the original physiological signal at the related point or time or during the related period of time with the predefined physiological threshold. If the amplitude change of the original physiological signal is smaller than or equal to the predefined physiological threshold, the detection accuracy of the physiological signal detector 18 may be not affected, and step S106 can be executed to output the original physiological signal generated by the physiological signal detector 18.

If the amplitude change of the original physiological signal is greater than the predefined physiological threshold, the detection accuracy of the physiological signal detector 18 may be worst, so that step S110 can be executed to compare duration of the amplitude change of the physiological signal exceeding the predefined physiological threshold with a predefined period of time. When the foresaid duration is smaller than or equal to the predefined period of time, an abnormal detection result of the physiological signal detector 18 may be decided as temporary error disturbance, and step S106 can be executed to output the original physiological signal generated by the physiological signal detector 18. When the foresaid duration is greater than the predefined period of time, the detection result of the physiological signal detector 18 is abnormal, and step S112 can be executed to optimize the original physiological signal for improving the detection accuracy of the physiological signal generated by the smart wearable device 10. Values of the predefined physiological threshold and the duration may designed due to the detection sensitivity of the physiological signal detector 18, and the detailed description is omitted herein for simplicity.

Step S112 can provide several types of optimization process. One of the optimization process can mark the original physiological signal within a specific period when the pressure value exceeds the predefined pressure threshold and the amplitude change of the physiological signal exceeds the predefined physiological threshold and the duration exceeds the predefined period of time, such as the abnormal physiological signal marked by the dashed rectangular box shown in FIG. 3, and then can further output a warning reminder relevant to the marked physiological signal, for informing the user that the abnormal physiological signal may not be considered. The warning reminder can be the visual warning, the audio warning or the tactile warning; therefore, the smart wearable device 10 may accordingly include the display panel, the speaker or the piezoelectric module, and depend on the design demand.

Another optimization process can also mark the original physiological signal within the specific period when the pressure value exceeds the predefined pressure threshold and the amplitude change of the physiological signal exceeds the predefined physiological threshold and the duration exceeds the predefined period of time, such as the abnormal physiological signal marked by the dashed rectangular box shown in FIG. 3, and the original physiological signal detected and stored before the foresaid specific period in step S100, such as a dashed elliptical box shown in FIG. 3, can be found from a database, and the abnormal physiological signal within the dashed rectangular box can be replaced by the normal physiological signal within the dashed elliptical box, so that the smart wearable device 10 can provide a reasonable and correct physiological detection result. That is to say, the smart wearable device 10 can compute a computation value of the physiological signal in the normal period, such as a mean value or a median value, to replace the abnormal physiological signal in the abnormal period.

It should be mentioned that the signal calibration method illustrated in FIG. 4 can be one embodiment of the smart wearable device 10. In other possible embodiments, when the pressure value exceeding the predefined pressure threshold is decided in step S104, step S112 may be directly executed to optimize the original physiological signal; further, when the amplitude change of the original physiological signal exceeding the predefined physiological threshold is decided in step S108, step S112 may be executed directly to optimize the original physiological signal. Thus, step S108 and step S110 can be optional steps and can be omitted in accordance with the design demand.

Moreover, the signal calibration method of the present invention can further compute duration of the pressure value being greater than the predefined pressure threshold in step S104. If the foresaid duration is smaller than or equal to the predefined period of time, the abnormal physiological signal of the physiological signal detector 18 may be generated due to the posture change of the user, and the physiological signal may be recovered into a normal mode when the posture of the user is changed. If the foresaid duration is greater than the predefined period of time, the smart wearable device 10 may be worn on the wrong place of the user; for example, the smart wearable device 10 may be designed to be worn as the ring on the ring finger, but is mistakenly worn on the middle finger, that is, the physiological signal detector 18 may continuously generate the abnormal physiological signal, and can output the warning reminder relevant to a situation that the duration of the pressure value exceeding the predefined pressure threshold is greater than the predefined period of time, so as to inform the user whether the smart wearable device 10 is worn on the wrong finger or the finger is pressed by a heavy object. A length of the predefined period of time can be manually set by the user, or can be a factory default value of the smart wearable device 10.

The acceleration detector 22 can be an optional element used to increase the detection accuracy of the physiological signal detector 18 with the pressure detector 20. For example, in step S104, when the pressure value is greater than the predefined pressure threshold and the acceleration value generated by the acceleration detector 22 which detects the detection area is greater than a predefined acceleration threshold, the signal calibration method can execute step S108 or step S112 optionally. A value of the predefined acceleration threshold can depend on the detection sensitivity of the acceleration detector 22 and operation habits of the user, and the detailed description is omitted herein for simplicity.

In conclusion, the smart wearable device of the present invention can utilize the detection results of the pressure detector and the acceleration detector to calibrate and/or optimize the physiological signal detected by the physiological signal detector; if the pressure value of the pressure detector and the acceleration value of the acceleration detector are at the high level, the smart wearable device may be set in an abnormal operation state, and the physiological signal generated within the related period can be marked as the abnormal result, or can be directly replaced by the computation value of the normal physiological signal. The pressure detector can be a capacitive pressure detector, a resistive pressure detector or a piezoelectric pressure detector; any pressure detector that can be disposed inside the case of the smart wearable device and provide the detection accuracy meeting the pressure standard can belong to a design scope of the present invention. Comparing to the prior art, the present invention can utilize both the pressure detector and the acceleration detector to optimize the detection result of the physiological signal detector, so as to effectively avoid the abnormal detection result caused by the user's posture, thus improving product credibility and reliability.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A smart wearable device having a signal calibration function and applied to a finger, a limb and/or a neck of a user, the smart wearable device comprising:
at least one physiological signal detector adapted to abut against a detection area of the user for detecting a physiological signal;
at least one pressure detector disposed around the at least one physiological signal detector and adapted to detect a pressure value of the detection area; and
an operation processor electrically connected with the at least one physiological signal detector and the at least one pressure detector, the operation processor being adapted to optimize the physiological signal when the pressure value exceeds a predefined pressure threshold.

2. The smart wearable device of claim 1, wherein the at least one pressure detector is disposed adjacent to an outer edge of the at least one physiological signal detector, so that the physiological signal and the pressure value of the detection area are detected by the at least one physiological signal detector and the at least one pressure detector in a respective and simultaneous manner.

3. The smart wearable device of claim 1, wherein the operation processor is adapted to further mark the physiological signal detected in a process of the pressure value exceeding the predefined pressure threshold, and output a warning reminder relevant to the marked physiological signal.

4. The smart wearable device of claim 1, wherein the operation processor is adapted to further replace the physiological signal detected in a process of the pressure value exceeding the predefined pressure threshold by the physiological signal detected before the process of the pressure value exceeding the predefined pressure threshold.

5. The smart wearable device of claim 1, wherein the operation processor is adapted to further optimize the physiological signal when the pressure value exceeds the predefined pressure threshold and amplitude change of the physiological signal exceeds a predefined physiological threshold.

6. The smart wearable device of claim 1, wherein the operation processor is adapted to further optimize the physiological signal when the pressure value exceeds the predefined pressure threshold and duration of amplitude change of the physiological signal exceeding a predefined physiological threshold conforms to a predefined period of time.

7. The smart wearable device of claim 1, wherein the operation processor is adapted to further output a warning reminder relevant to the predefined pressure threshold when duration of the pressure value exceeding the predefined pressure threshold conforms to a predefined period of time.

8. The smart wearable device of claim 1, wherein the smart wearable device further comprises a plurality of physiological signal detectors and a plurality of pressure detectors, each of the plurality of pressure detectors is disposed adjacent to the outer edge of a corresponding physiological signal detector as a pair, for simultaneously detecting the physiological signal and the pressure value of a corresponding detection area.

9. The smart wearable device of claim 1, wherein the smart wearable device further comprises an acceleration detector electrically connected with the operation processor and adapted to detect an acceleration value of the detection area, the operation processor is adapted to further optimize the physiological signal when the pressure value exceeds the predefined pressure threshold and the acceleration value exceeds a predefined acceleration threshold.

10. A signal calibration method of calibrating a physiological signal detected by a smart wearable device wore on a detection area of a user, the signal calibration method comprising:
analyzing a pressure value of the detection area detected by at least one pressure detector of the smart wearable device; and
optimizing the physiological signal of the detection area detected by at least one physiological signal detector of the smart wearable device when the pressure value exceeds a predefined pressure threshold, for acquiring preferred accuracy of the physiological signal.

11. The signal calibration method of claim 10, further comprising:
marking the physiological signal detected in a process of the pressure value exceeding the predefined pressure threshold; and
output a warning reminder relevant to the marked physiological signal.

12. The signal calibration method of claim 10, further comprising:
storing the physiological signal detected by the at least one physiological signal detector before a process of the pressure value exceeding the predefined pressure threshold; and
replacing the physiological signal detected by the at least one physiological signal detector in the process of the pressure value exceeding the predefined pressure threshold by the said physiological signal detected before the said process.

13. The signal calibration method of claim 10, further comprising:
optimizing the physiological signal when the pressure value exceeds the predefined pressure threshold and amplitude change of the physiological signal exceeds a predefined physiological threshold.

14. The signal calibration method of claim 10, further comprising:
optimizing the physiological signal when the pressure value exceeds the predefined pressure threshold and duration of amplitude change of the physiological signal exceeding a predefined physiological threshold conforms to a predefined period of time.

15. The signal calibration method of claim 10, further comprising:
outputting a warning reminder relevant to the predefined pressure threshold when duration of the pressure value exceeding the predefined pressure threshold conforms to a predefined period of time.

16. The signal calibration method of claim 10, further comprising:
optimizing the physiological signal when the pressure value exceeds the predefined pressure threshold and an acceleration value of the detection area detected by an acceleration detector of the smart wearable device exceeds a predefined acceleration threshold.

17. The signal calibration method of claim 10, wherein the at least one pressure detector is disposed adjacent to an outer edge of the at least one physiological signal detector, so the physiological signal and the pressure value of the detection area are detected by the at least one physiological signal detector and the at least one pressure detector in a respective and simultaneous manner.

18. The signal calibration method of claim 10, wherein the smart wearable device further comprises a plurality of physiological signal detectors and a plurality of pressure detectors, each of the plurality of pressure detectors is disposed adjacent to the outer edge of a corresponding physiological signal detector as a pair, for simultaneously detecting the physiological signal and the pressure value of a corresponding detection area.
